(19) United States
(12) Patent Application Publication
(10) Pub. No.: US 2003/0170282 A1
(43) Pub. Date: Sep. 11, 2003

(54) Method of skin protection during hair treatment

(76) Inventor: David K. Walz, Stone Mountain, GA (US)

Correspondence Address: Walter A. Rodgers, 880 North Island Drive, Atlanta, GA, 30327, US

(21) Appl. No.: 10/375,875

(22) Filed: Feb. 26, 2003

Related U.S. Application Data
(60) Provisional application No. 60/361,489, filed on Mar. 5, 2002.

(51) Int. Cl. A61K 7/06

(52) U.S. Cl. 424/401

(57) ABSTRACT

A method of applying a base cream from a pressurized container around an individual's head by applying a thin bead of base cream adjacent the individual's hairline and in some cases directly on the scalp between the ribbon layers of relaxer that are applied to new growth.

The benefits under 35 U.S.C.119 are claimed of provisional application No. 60/361,489 filed Mar. 5, 2002.

### BACKGROUND OF THE INVENTION

In the process of relaxing, straightening or applying a permanent wave to an individual's hair, harsh chemicals are utilized which can cause severe damage if they come into contact with an individual's skin. Generally, a thin ribbon of base cream, such as petroleum jelly, neutralizer, gel, etc., is applied around the base of the hairline to protect the skin from the treatment chemicals.

Typically, the base cream is applied by using a finger, gloved hand, back side of a comb, spatula-type instrument or a brush to manually apply the base cream around the circumference of the individuals head just below the hairline. In the case of individuals with sensitive skin, cream is also applied where the hair is parted during the chemical application process.

This procedure requires constant back and forth motion between the base cream container and the skin which is quite time consuming and results in a imprecise application of the cream. In addition, the unopened container is exposed to the elements and resultant contamination.

### SUMMARY OF THE INVENTION

By this invention, a thin bead of base cream is applied to an individual's head adjacent the hairline by dispensing base cream in a container under pressure through a spout such that a thin bead of cream is applied immediately adjacent the hairline around the entire circumference of the hairline. In addition, with persons with extremely sensitive skin, this invention allows for the application of base cream directly to the skin, between the ribbons of relaxer, as it is being applied to new growth which thereby reduces the amount of relaxer in contact with the scalp.

### BRIEF DESCRIPTION OF THE DRAWINGS

In the drawings,

FIG. 1 is an elevational view showing the base cream container according to this invention; and

FIG. 2 is a perspective view showing application of the base cream.

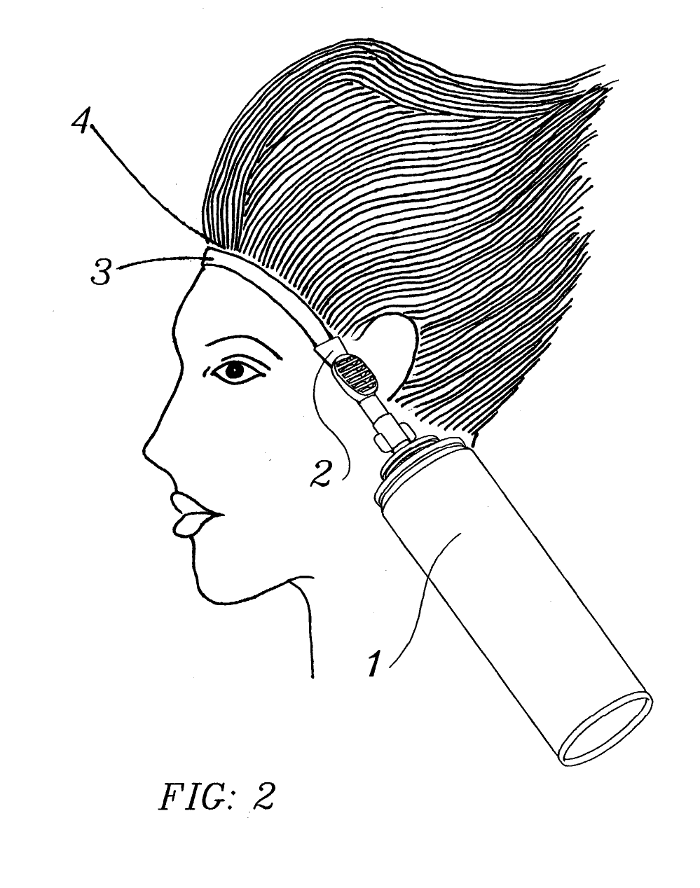

### DETAILED DESCRIPTION OF THE INVENTION

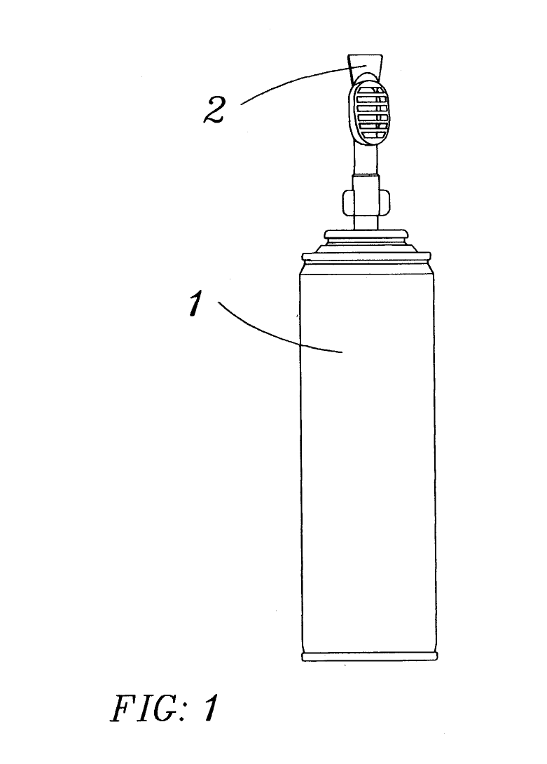

In the drawings and with particular reference to FIG. 1, the numeral 1 designates a pressurized container for dispensing a protective cream barrier according to this invention. Pressurized container 1 is used for both storing and dispensing the base cream, typically comprising petroleum jelly, neutralizer, gel, etc. The creme is contained within container 1 in an internal bag (not shown) to isolate the cream and then the base cream is dispensed through spout 2, as is well known. Alternatively, a piston barrier between the base cream (chemical) and the propellant can be utilized in lieu of the bag in a can approach. In addition, depending on the make-up of the base cream, the base cream and the propellant can occupy the same space/compartment.

By this invention, the base cream is dispensed though spout 2 so that a precise ribbon or bead of base cream 3 is applied immediately adjacent hairline 4. Bead 3 is applied across the forehead, around the ear and around the back of the individual's head all the way around the other side of the head to the point where the bead was started so that a complete 360 degree barrier is provided between the individual's hair and skin.

Therefore, by this invention, a beautician can apply a thin ribbon of protective base cream in a much shorter time than the conventional manual method. The result is also a protective bead which is much more precisely applied than known methods and results in cost economy and greater uniformity.

1. A method of skin protection during chemical treatment of an individual's hair comprising the steps of dispensing a base cream from a pressurized container through a spout, applying said cream to an individual's skin immediately adjacent the hairline, and continuously applying said cream around the entire hairline of the individual.

2. A method according to claim 1 wherein said base cream is applied in a thin bead.

3. A method according to claim 2 wherein said bead is uniform.

4. A method according to claim 1 wherein ribbons of relaxer are applied to new hair growth and wherein said base cream is applied to the scalp between said ribbons.
